(19) United States
(12) Patent Application Publication
(10) Pub. No.: US 2026/0049045 A1
(43) Pub. Date: Feb. 19, 2026

(54) Amplificative Ethylene Detection by Activation of Latent Olefin Metathesis Catalysts and Systems

(71) Applicant: University of Denver, Denver, CO (US)

(72) Inventors: Brian Michel, Denver, CO (US); Brady Worrell, Denver, CO (US); Autumn Giger, Conifer, CO (US); Jaiden Voldrich, Conifer, CO (US)

(73) Assignee: University of Denver, Denver, CO (US)

(21) Appl. No.: 19/301,822

(22) Filed: Aug. 15, 2025

Related U.S. Application Data
(60) Provisional application No. 63/683,597, filed on Aug. 15, 2024.

(51) Int. Cl. C07C 11/04; C07C 6/04; G01N 21/64; G01N 27/26

(52) U.S. Cl. CPC C07C 11/04; C07C 6/04; G01N 21/64; G01N 27/26; C07C 2523/46

(57) ABSTRACT

The present disclosure provides methods and systems for ethylene detection, the methods and systems including activation of an olefin metathesis catalyst by initial ethylene molecules to produce signal molecules and secondary ethylene molecules, where the ethylene includes the initial ethylene molecules and the secondary ethylene molecules, and detecting the ethylene.

### REFERENCE TO RELATED APPLICATIONS

The present application claims the benefit of U.S. Prov. Pat. App. No. 63/683,597, filed on 2024 Aug. 15 and titled “Amplificative Ethylene Detection by Activation of Latent Olefin Metathesis Catalysts and Systems,” which is incorporated hereby in its entirety by reference.

### SPONSORED RESEARCH AND DEVELOPMENT

This invention was made with government support under R35GM150937 awarded by the National Institutes of Health and 2023-67018-39467 awarded by the US Department of Agriculture. The government has certain rights in the invention.

### FIELD OF THE DISCLOSURE

The present disclosure relates generally to ethylene detection and more specifically, but not by way of limitation, methods and systems used for ethylene detection using molecular approaches.

### DESCRIPTION OF RELATED ART

The importance of ethylene as a signaling molecule in plants has driven interest in developing molecular strategies for its detection because ethylene, although playing a central role in the natural ripening process of climacteric fruit, can also trigger deterioration of produce. Premature ripening, spoilage, produce damage by pests and diseases are some of the primary culprits for food waste in the US. Additionally, short shelf-life and blemished produce deter consumers. Thus, the detection of ethylene is of particular interest for plant and food supply chain management. Further areas of potential use of ethylene detections systems include applications ranging from consumer test kits to chemical biology; for example, monitoring ethylene leaks at industrial sites and optimization of bioethylene production, among others.

### BRIEF DESCRIPTION OF DRAWINGS

FIG. 1 illustrates prior approaches to stoichiometric signal transduction.

FIG. 2 is a diagram illustrating an approach to amplificative sensing for ethylene detection, in accordance with various embodiments disclosed herein.

FIG. 3A is an illustrative example showing activation of latent metathesis catalyst, in accordance with various embodiments disclosed herein.

FIG. 3B is an illustrative diagram showing an example of the general concept of signal amplification by ruthenium (Ru) catalysis, in accordance with various embodiments disclosed herein.

FIG. 3C illustrates steps of a ring closing metathesis (RCM) process, in accordance with various embodiments disclosed herein.

FIG. 3D illustrates steps of a ring opening metathesis polymerization (ROMP) process, in accordance with various embodiments disclosed herein.

FIG. 4 shows an illustrative diagram of autocatalytic behavior involved in various embodiments disclosed herein.

FIG. 5 is an illustrative graph showing kinetic profiles for the RCM of N,N-diallyl-4-methylbenzenesulfonamide (TsN(allyl)₂) with different amounts of ethylene (C₂H₄), in accordance with various embodiments disclosed herein.

FIG. 6 is an illustrative graph showing kinetic profiles for the substrate, showing substrate consumption as a function of time, in accordance with various embodiments disclosed herein.

FIG. 7 is an illustrative graph showing kinetic profiles for dissolved ethylene over time, in accordance with various embodiments disclosed herein.

FIG. 8A shows an exemplary set of catalyst candidates evaluated in screening tests, in accordance with various embodiments disclosed herein.

FIG. 8B illustrates an RCM process for ProU and Pro3MU, in accordance with various embodiments disclosed herein.

FIG. 8C illustrates a relay RCM process for DQ-1, in accordance with various embodiments disclosed herein.

FIG. 9 shows an illustrative bar graph summarizing results of a catalyst screening process with Pro3MU, in accordance with various embodiments disclosed herein.

FIG. 10 shows an illustrative bar graph summarizing results of a catalyst screening process with DQ-1, in accordance with various embodiments disclosed herein.

FIG. 11A is an illustrative graph comparing relative substrate response over time, in accordance with various embodiments disclosed herein.

FIG. 11B is an illustrative graph comparing fluorescence turn-on values for DQ-1 over time, in accordance with various embodiments disclosed herein.

FIG. 12 is an illustrative graph summarizing the evaluation results of extended Pro3MU latency with 12 catalysts, in accordance with various embodiments disclosed herein.

FIG. 13 is an illustrative graph summarizing the influence of temperature on DQ-1 latency, in accordance with various embodiments disclosed herein.

FIG. 14 is an illustrative bar graph summarizing the results of the evaluation of selectivity for C₂H₄ compared to other substrates, in accordance with various embodiments disclosed herein.

FIG. 15A shows an illustrative line region for limit of detection experiments with injected ethylene in accordance with various embodiments disclosed herein.

FIG. 15B shows an illustrative line region for limit of detection experiments with injected ethylene in accordance with various embodiments disclosed herein.

FIG. 15C shows illustrative combined regions of linearity for limit-of-detection experiments with injected ethylene in accordance with various embodiments disclosed herein.

FIG. 15D shows illustrative fluorescence intensity over time at low volumes of ethylene for limit-of-detection experiments with injected ethylene in accordance with various embodiments disclosed herein.

FIG. 16A is an illustrative example flow system for larger volume dilute ethylene sampling in accordance with various embodiments disclosed herein.

FIG. 16B shows an illustrative demonstration of fluorescence response from a flow system in accordance with various embodiments disclosed herein.

FIG. 17A shows an experiment demonstrating endogenous ethylene detection from apples in accordance with various embodiments disclosed herein.

FIG. 17B is an illustrative diagram showing the influence of 1-aminocyclopropane-1-carboxylic acid (ACC) and pyrazinamide (PZA) on ethylene production, in accordance with various embodiments disclosed herein.

FIG. 17C is an illustrative bar graph of average fluorescence response to increasing amounts of ACC, in accordance with various embodiments disclosed herein.

FIG. 17D is an illustrative bar graph of average fluorescence response to inhibition of ACC oxidation (ACO) with PZA, in accordance with various embodiments disclosed herein.

FIG. 18 shows an illustrative proposed route to ethylene from lipid peroxides, in accordance with various embodiments disclosed herein.

FIG. 19 is an illustrative closed loop sampling set-up employed in lipid peroxidation experiments in accordance with various embodiments disclosed herein.

FIG. 20 is an illustrative bar graph showing the detection of ethylene from peroxidation and fragmentation of linoleic acid and linolenic acid, in accordance with various embodiments disclosed herein.

FIG. 21A shows normalized emission over time for Pro3MU for concentrations of catalyst and substrate, in accordance with various embodiments disclosed herein.

FIG. 21B shows normalized emission over time for Pro3MU for concentrations of catalyst and substrate, in accordance with various embodiments disclosed herein.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION

Features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, where like reference numerals designate corresponding parts in the various figures. It is to be expressly understood, however, that the drawings are for the purpose of illustration and description only and are not intended as a definition of the limits of this disclosure. As used in the specification and in the claims, the singular form of ‘a,’ ‘an,’ and ‘the’ include plural referents unless the context clearly dictates otherwise.

In the description herein, various ranges and/or numerical limitations can be expressly stated below. It should be recognized that unless stated otherwise, it is intended that endpoints are to be interchangeable. Further, any ranges include iterative ranges of like magnitude falling within the expressly stated ranges or limitations.

Furthermore, various modifications can be made within the scope of the invention as herein intended, and embodiments of the invention can include combinations of features other than those expressly claimed. In particular, flow arrangements other than those expressly described herein are within the scope of the invention.

Regarding claim transitional terms or phrases, the transitional term “comprising”, which is synonymous with “including,” “containing,” “having” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter composition or method to which the term is applied. For example, a feedstock consisting of a material A can include impurities typically present in a commercially produced or commercially available sample of material A. When a claim includes different features and/or feature classes (for example, a method step, feedstock features, and/or product features, among other possibilities), the transitional terms comprising, consisting essentially of, and consisting of apply only to the feature class that is utilized and it is possible to have different transitional terms or phrases utilized with different features within a claim. For example, a method can comprise several recited steps (and other non-recited steps) but utilize a catalyst system preparation consisting of specific steps can utilize a catalyst system comprising recited components and other non-recited components.

Within this specification, use of “comprising” or an equivalent expression contemplates the use of the phrase “consisting essentially of,” “consists essentially of,” or equivalent expressions as alternative embodiments to the open-ended expression. Additionally, use of “comprising” or an equivalent expression or use of “consisting essentially of” in the specification contemplates the use of the phrase “consisting of,” “consists of,” or equivalent expressions as an alternative to the open-ended expression or middle ground expression, respectively. For example, “comprising” should be understood to include “consisting essentially of,” and “consisting of” as alternative embodiments for the aspect, features, and/or elements presented in the specification unless specifically indicated otherwise.

While compositions and methods are described in terms of “comprising” various components or steps, the compositions and methods can also “consist essentially of” or “consist of” the various components or steps.

For any particular compound disclosed herein, the general structure or name presented is also intended to encompass all structural isomers, conformational isomers, and stereoisomers that can arise from a particular set of substituents, unless indicated otherwise. Thus, a general reference to a compound includes all structural isomers unless explicitly indicated otherwise. Additionally, the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that can arise from a particular set of substituents.

Processes, systems, and/or reaction systems described herein can utilize steps, features, compounds and/or equipment which are independently described herein. The processes, systems, and/or reaction systems described herein may or may not utilize step identifiers (e.g., 1), 2), etc., a), b), etc., i), ii), etc., or first, second etc., among others), feature identifiers (e.g., 1), 2), etc., a), b), etc., i), ii), etc., or first, second etc., among others), and/or compound and/or composition identifiers (e.g., 1), 2), etc., a), b), etc., i), ii), etc., or first, second etc., among others). However, it should be noted that processes, systems, and/or reaction systems described herein can have multiple steps, features (e.g. reagent ratios, formation conditions, among other considerations), and/or multiple compounds and/or composition using no descriptor or sometimes having the same general identifier. Consequently, it should be noted that the processes, systems, and/or reaction systems described herein can be modified to use an appropriate step or feature identifier (e.g., 1), 2), etc., a), b), etc., i), ii), etc., or first, second etc., among others), feature identifier (e.g., 1), 2), etc., a), b), etc., i), ii), etc., or first, second etc., among others), and/or compound identifier (e.g., first, second, etc.) regardless of step, feature, and/or compound identifier utilized in the a particular aspect and/or embodiment described herein and that step or feature identifiers can be added and/or modified to indicate individual different steps/features/compounds utilized within the processes, systems, and/or reaction systems without detracting from the general disclosure.

According to various example aspects of the present disclosure, some embodiments are directed towards methods for ethylene detection. In some embodiments, the methods can include activation of an olefin metathesis catalyst by initial ethylene molecules to produce signal molecules and secondary ethylene molecules, where the ethylene includes the initial ethylene molecules and the secondary ethylene molecules; and detecting the ethylene.

Various embodiments include where the detecting the ethylene is based on the secondary ethylene molecules. Some aspects include where the detecting the ethylene is further based on self-propagation.

In certain aspects, the methods further include in-situ generation of latent units of the olefin metathesis catalyst that performs multiple cycles of substrate conversion to obtain an amplified signal of the ethylene. In some embodiments, methods include where an amount of the amplified signal corresponds to an amount of the secondary ethylene molecules.

In some aspects, more of the signal molecules are produced than the secondary ethylene molecules.

In further aspects, the methods include converting, using ring closing metathesis, non-fluorescent molecules to fluorescent molecules along with at least some of the secondary ethylene molecules. Some aspects include where the secondary ethylene molecules generate additional ethylene, and where the additional ethylene activates latent units of the olefin metathesis catalyst to provide an autocatalysis process.

Various embodiments include where the substrate conversion occurs by at least one of ring closing metathesis reactions, ring opening metathesis polymerization reactions, cross metathesis reactions, and acyclic diene metathesis polymerization reactions.

Some embodiments further include signal transduction during the substrate conversion. Various aspects include where the signal transduction occurs via at least one of profluorescent ring closing metathesis reactions and induced polymerization reactions.

Various aspects further include catalytic signal transduction using at least one mechanism including: fluorescence reactions, colorimetric reactions, polymerization, and electrochemical reactions.

Some embodiments further include after the activation, initiating ring opening metathesis polymerization using latent units of the olefin metathesis catalyst.

Further embodiments can include polymerization, where the polymerization accelerates the production of the secondary ethylene molecules. In certain aspects, the polymerization is translated into at least one other signal transduction mechanisms including fluorescence reactions, luminescence reactions, and colorimetric reactions.

According to various example aspects of the present disclosure, some embodiments are directed towards systems for ethylene detection, including providing an olefin metathesis catalyst; activating the olefin metathesis catalyst using initial ethylene molecules to produce signal molecules and secondary ethylene molecules, where the ethylene includes the initial ethylene molecules and the secondary ethylene molecules; and detecting the ethylene.

According to various example aspects of the present disclosure, some embodiments are directed towards methods for ethylene amplification, including activation of an olefin metathesis catalyst by initial ethylene molecules to produce signal molecules and secondary ethylene molecules.

In various aspects, embodiments further include detecting ethylene after the activation, where the detecting the ethylene is based on the secondary ethylene molecules. Some aspects include where the detecting the ethylene is further based on self-propagation.

Some embodiments further include in-situ generation of latent units of the olefin metathesis catalyst that performs multiple cycles of substrate conversion to obtain an amplified signal of the ethylene.

While ethylene has long been known as an important plant hormone, it has also been demonstrated to be produced from lipid peroxides in mammals as a result of oxidative stress that is hallmark of numerous diseases. That is, ethylene may serve as an important biomarker of oxidative stress from lipid peroxidation. The endogenous production of small hydrocarbons in mammals has been well documented for decades. Molecules such as ethane, pentane, and ethylene have been detected from mammalian systems. In particular, the predominant endogenous source of ethylene is lipid peroxides, which arise from the oxidation of polyunsaturated fatty acids such as linoleic and arachidonic acid through radical abstraction of the relatively weak doubly allylic C—H bonds by reactive free radicals. A series of fragmentations reactions can provide a number of small hydrocarbons, predominately ethylene. Since lipid peroxidation is associated with oxidative stress, these biologically unusual species represent biomarkers of oxidative stress, which is associated with a myriad of diseases. Ethylene detection in exhaled breath has been demonstrated as a biomarker of oxidative stress in mammals.

Compared to saturated hydrocarbons, the presence of an alkene in ethylene enables oxidation to carcinogenic ethylene oxide or ethylene could bind to a metalloprotein in yet undiscovered signaling pathways. Additionally, there is evidence that ethylene can be produced by non-radical pathways in the liver and from the gastrointestinal microbiome. These other possible sources of endogenous ethylene are poorly defined and the fact that these routes are rarely discussed in mammalian systems highlights how little is still known about endogenous ethylene production.

The volatility of ethylene and spectral signatures has made analysis of levels in exhaled breath a non-invasive method for monitoring oxidative stress in a variety of situations including bacterial infection, UV light exposure, presence of redox active metals such as nickel, stresses caused by procedures such as hemodialysis in patients with renal failure, and during cardiac surgery. Therefore, tools capable of detecting ethylene provide a means for studying the role of ethylene as a biomarker of oxidative stress.

Gas chromatography (GC) is a classic, laboratory-based method of ethylene detection. However, GC systems are expensive, require pre-processing of samples, and generally not convenient for most use case scenarios outside of a laboratory setting. As another example, laser photoacoustic spectroscopy (LPAS) is an incredibly sensitive technique that has generally supplanted gas chromatography as the preferred method for exhaled breath analysis of ethylene. Ethylene detection to assess oxidative stress in humans and other mammals have largely been accomplished in the headspace above a cell culture or via analyzing exhaled breath with LPAS instruments.

However, to achieve high sensitivity detection, exhaled breath needs to be scrubbed of CO₂ and H₂O to simplify the matrix. Further LPAS systems are expensive and specialized instruments that are often custom-built, precluding widespread adoption. Moreover, the analysis of a simplified matrix in the gas phase obviously limits the spatially resolved study of ethylene at the cellular level. Additionally, the operational requirements of the LPAS instruments inherently limit the study of ethylene at the microscopic level, hampering the use of standard cell assays and high throughput approaches.

Another option that is currently available is electrochemical sensing, in which detected changes in electrical signal in specialized sensor circuitry correspond to the presence of ethylene. While usually highly portable, electrochemical sensors have limited shelf life, particularly in high ethylene concentration environments.

The development of technologies to detect ethylene at the microscopic level would have broad impacts on the ability to study production and possible signaling roles of ethylene by the greater biomedical community. The current level of incomplete knowledge of ethylene production in mammals demonstrates that there is a clear unmet need for tools with high spatial and temporal resolution that can detect this intriguing biomarker.

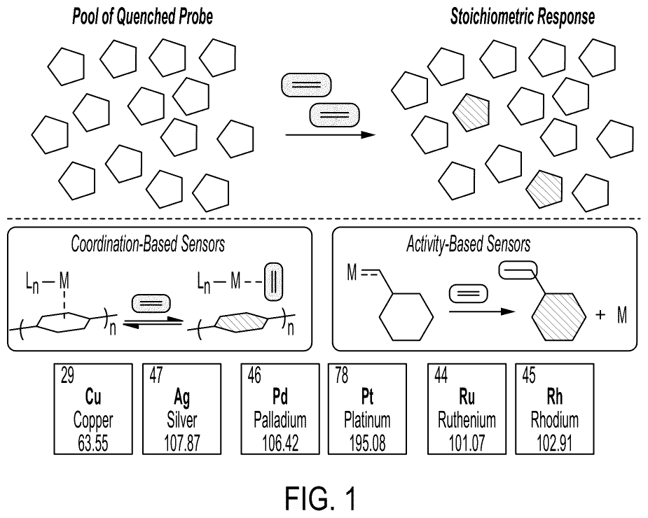

There are a number of current molecular approaches to detecting ethylene; However, all of these existing methods are limited by a 1:1 detection to signal output. Further, some complexes described are prone to false positive results resulting from decomposition. For example, FIG. 1 shows concepts used in prior techniques related to stoichiometric signal transduction. Currently available molecular approaches to ethylene detection include coordination-based sensors, where detection occurs via the coordination of ethylene to a metal center, typically silver (Ag) or copper (Cu), with signal transduction via optical, chemiresistive, and gravimetric responses.

Another approach is an Activity-Based Sensor (ABS) for ethylene detection. By utilizing transition metal mediated reactions (olefin metathesis or migratory insertion) for analyte detection, high levels of selectivity and sensitivity may be achieved by transition state energetics by ABS systems. One exemplary approach is based on the stoichiometric reaction of a Hoveyda-Grubbs 2nd generation catalyst complex appended with a fluorophore on the chelating isopropoxy ligand. In this ABS system, a reaction occurs following ethylene coordination (e.g., olefin metathesis).

Additional examples of ABS probes for ethylene have been reported since, including strategies employing protein-ABS adducts and cyclorhodinated complexes. An earlier example likely proceeds via a Wacker oxidation, such as one approach that is catalytic in palladium (Pd). The current research paradigm in the field of transition metal based ABSs largely involves modest and/or unfocused approaches to structure modifications.

A long-standing challenge in applying transition metals to biological systems is the high concentration of competing ligands. A creative approach has been to protect the metal center in a protein binding pocket via steric and charge-based repulsion strategies such as the reported artificial metalloenzyme (ArM) approaches. While ArMs have made inspiring advancements and are a viable technological approach to transition metal-based ABSs, the design is laborious, preparation of complicated ligands is technically demanding, and discrete characterization hinders rational improvements.

In such examples, signal transduction arises from a stoichiometric event (coordination or coordination-then-reaction). While there is a prior technique that reuses the palladium, signal transduction remains stoichiometrically related. Thus, there is a need for an improved approach for high sensitivity detection of ethylene applicable to a variety of measurement conditions.

While there are advantages to defined metal complexes that provide a stoichiometric ethylene detection (e.g., one-to-one), previously-known systems did not use the power of catalysis to achieve signal amplification. Various approaches to ethylene detection described herein are based on activation of latent olefin metathesis catalysts, resulting in a signal output that is advantageously amplified by both catalysis and in some cases self-propagation.

Amplified detection systems have been developed to provide significant signal output in the presence of limited target analyte. One amplification system is the polymerase chain reaction (PCR) used in molecular sensing, which can be used to amplify the target DNA sequences. Transition metal catalysis (e.g., a system for detecting trace palladium) has also been used for amplificative detection. However, such amplified detection approaches have not been applied in prior techniques for ethylene detection.

An exemplary aspect of the catalyst activation approach described in embodiments herein is that, while certain olefin metathesis precatalysts are unreactive with substrates due to the steric environment, ethylene, due to its small size, can react with these complexes. In various aspects, the inventors of the present disclosure have determined that the stoichiometry of Ruthenium (Ru) complexes reacting with ethylene is advantageously useful for the catalyst activation approach.

Such amplificative approaches may be advantageous for a variety of applications, such as ethylene detection in the food supply chain, exhaled breath, industrial settings such as chemical plants, and biological sources such as bacteria. Furthermore, a sensitive, molecular approach to ethylene detection may be useful in a variety of settings, including and not limited to agricultural warehouses, supply chain companies, and biomedical corporations by researchers in agricultural, energy, and health fields, for instance.

In various aspects, it is described herein that ethylene activation of latent olefin metathesis catalysts may be used as the basis of ethylene detection with signal amplification. While some studies on latent olefin metathesis catalysts focus on activation by heat and/or light, the present disclosure advantageously recognizes and validates that, in embodiments, the activation of latent olefin metathesis catalysts by ethylene is possible and useful for ethylene detection.

For example, one approach notes an initial lag phase prior to rate acceleration for the sterically encumbered nG-SIPr-I2, which is ablated in the presence of ethylene. These results are recognized in various embodiments disclosed herein, for example an initially slow catalyst is likely converted into a more active species (e.g., Ru=CH₂) following the first few turnovers of substrate.

Similarly, another approach describes replacement of the chloride ligands bulky iodides resulting in cis-Ru-SPh-I2. This complex demonstrated excellent latency with no polymerization of cyclooctene (COE) observed over 24 hours at 80° C. However, upon addition of diethyldiallylmalonate (DEDAM) the reaction mixture readily polymerized. That is, this example describes a ring opening metathesis polymerization (ROMP) process, which results in a polymer as a product.

It is recognized that in embodiments of the present disclosure, cycloalkenes may be sterically blocked from coordination to benzylidene species derived from the precatalyst; however, the catalyst could perform ring opening metathesis polymerization (ROMP) of DEDAM. The resultant ethylene adduct is a highly reactive methylidene capable of catalytic ROMP presumably due to reduced steric encumbrance. Instead, ring closing metathesis (RCM) may be used to convert terminal diene molecules into cycloalkene molecules such that ethylene may be considered a byproduct of RCM. That is, appropriately designed non-fluorescent molecules can undergo RCM to be converted into fluorescent molecules along with ethylene, such that initial ethylene molecules (e.g., detected ethylene) generate secondary ethylene molecules (e.g., additional ethylene), which can feedback to activate more of the latent catalyst pool (e.g., latent olefin metathesis catalyst not used by the initial production of the additional ethylene) to further amplify the detection of ethylene in an autocatalysis process. In other words, in various embodiments, latent catalysts can be initiated by ethylene, and the detected ethylene generates a product, which can result in signal transduction.

Systems for ethylene detection described herein can include embodiments that detect ethylene in low amounts by activation of latent olefin metathesis catalyst by ethylene, which results in the in-situ generation of an active catalyst that can perform multiple cycles of substrate conversion resulting in an amplified signal (e.g., more signal molecules may be produced than ethylene molecules detected). In various embodiments disclosed herein, amounts of ethylene detected may be below 100 nanoliters (nL). The substrate conversion can occur by ring closing metathesis reactions, ring opening metathesis polymerization reactions, cross metathesis reactions, and acyclic diene metathesis polymerization reactions. For example, various embodiments disclosed herein show that Pro3MU and DQ-1 undergo ring closing metathesis.

A chemical structure for 5-hydroxy-2-vinylphenyl methacrylate (Pro3MU) is shown in Chemical Structure 1:

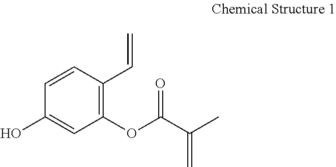

A chemical structure for 1-(2,4-dinitrophenyl)-2,5-dihydro-1H-pyrrole (Q-1) is shown in Chemical Structure 2:

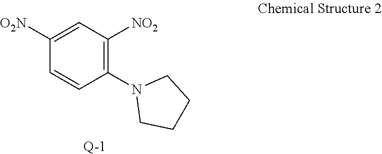 [SMILES: O=[N+]([O-])c1ccc(N2CCCC2)c([N+](=O)[O-])c1 ; "Q-1"]

A chemical structure for 5-((2,5-dihydro-1H-pyrrol-1-yl) sulfonyl)-N,N-dimethylnaphthalen-1-amine (D-1) is shown in Chemical Structure 3:

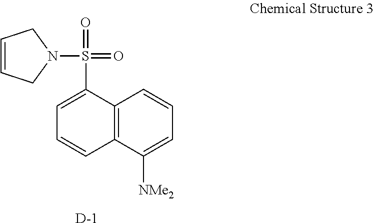

A chemical structure for (E)-N-allyl-N-(4-(allyl(2,4-dinitrophenyl)amino) but-2-en-1-yl)-5-(dimethylamino) naphthalene-1-sulfonamide (DQ-1) is shown in Chemical Structure 4:

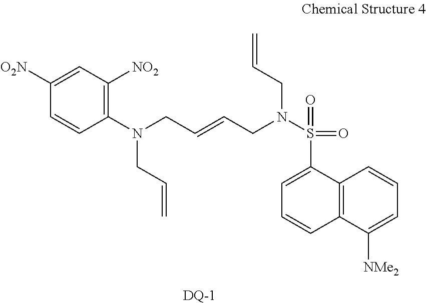

In some aspects, an acyclic diene can be converted into a cyclic alkene and ethylene, and the cyclic alkene may have optical properties (e.g., fluorescence) that enable signal transduction. In some embodiments, for ring opening metathesis polymerization, a cyclic alkene can be converted into a polyolefin product. A signal can be generated from this polymerization process via the various methods disclosed herein (e.g., optical, electrochemical, etc.). In various aspects, for cross metathesis reactions, two acyclic alkenes can undergo metathesis to generate a new acylic alkene and ethylene. In some aspects, for acyclic diene metathesis polymerization reactions, an acyclic diene can react with another acylic diene sequentially resulting in a polymer.

Signal transduction is possible via multiple outputs including profluorescent ring closing metathesis reactions or induced polymerization reactions. In particular, substrates may be designed to provide catalytic signal transduction after reaction by a variety of mechanisms including, and not limited to, fluorescence, colorimetric, polymerization, and electrochemical reactions. Further, this concept of the activation of a latent catalyst followed by RCM may be expanded to other signal transduction processes, such as colorimetric and electrochemical reactions. For example, in colorimetric reactions, the product of ring closing metathesis can absorb light at a different wavelength than the substrate and this can result in a color change. In a further example, polymerization reactions result in a more viscous solution. In a still further example, molecules can be incorporated into the solution that change color based on the solution viscosity. For example, in electrochemical reactions with ring closing metathesis, ring opening metathesis polymerization reactions, cross metathesis reactions, and acyclic diene metathesis polymerization reactions, these can result in products with extended or altered pi conjugation, which can result in a change in the oxidation/reduction potential of the molecule and alter their ability to act as electrochemical dopants. In a further example, polymerization reactions result in a more viscous solution. In a still further example, charge carries dissolved in the polymer solution can have restricted mobility and therefore a reduced current may be observed.

In various embodiments, the latent catalyst may be used to initiate ring opening metathesis polymerization (ROMP). In this case, while ethylene is not a byproduct of the reaction, other aspects of the polymerization reaction may act to accelerate the reaction rate, such as exhibited in the generation of heat. The polymerization reaction may also itself be a readily observable signal transduction, exhibited as a change in the physical property of the material (e.g., viscosity, hardness, etc.). The polymerization reaction may also be translated into other signal transduction mechanisms, such as fluorescence, luminescence, and colorimetric reactions.

In some aspects, some examples of polymerization reactions disclosed herein can use monomers with fluorophore attached that shift their emission wavelength in the tangled polymer network. In further examples, polymerization reactions can use two monomers with appropriately selected fluorophores attached that undergo an energy transfer process only upon polymerization. This can provide a fluorescence response when exciting using the appropriate wavelength light.

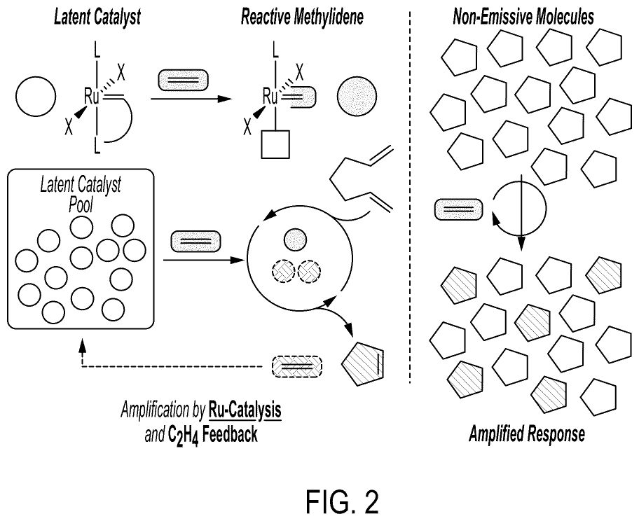

FIG. 2 shows a diagram illustrating an approach to amplificative sensing for ethylene detection, in accordance with various embodiments disclosed herein. Unlike prior methods, which are limited due to the stoichiometric nature of ethylene detection (e.g., one ethylene detection event results in one signal event), various embodiments described herein may result in multiple signal events for each ethylene detection event.

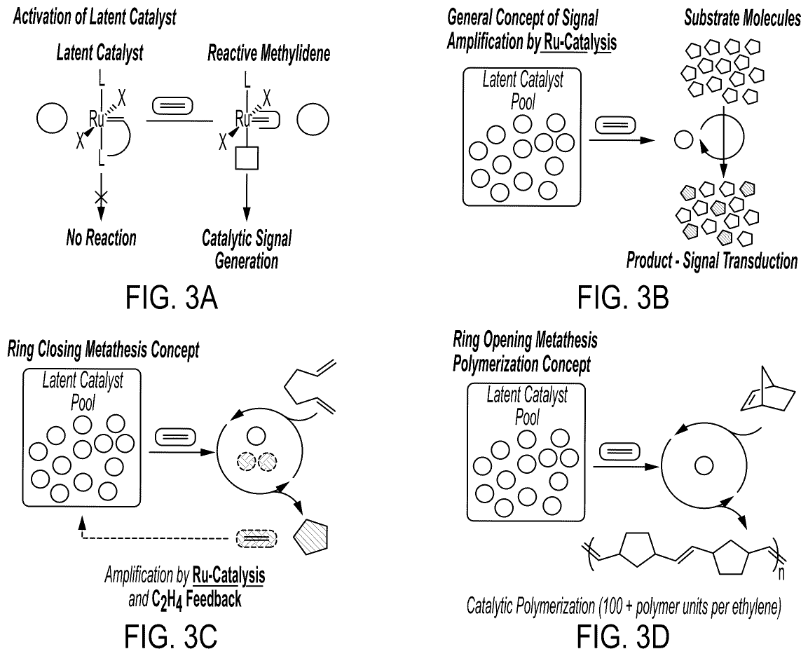

Some exemplary embodiments of the present disclosure specifically for RCM/fluorescence are illustrated, for example, in FIGS. 3A-C. In some aspects, FIG. 3A shows an illustrative example showing activation of latent metathesis catalyst, in accordance with various embodiments disclosed herein. FIG. 3B shows an illustrative diagram showing an example of the general concept of signal amplification by ruthenium (Ru) catalysis, in accordance with various embodiments disclosed herein. FIG. 3C illustrates steps of a ring closing metathesis (RCM) process, in accordance with various embodiments disclosed herein.

In various embodiments, to realize an amplified detection of ethylene, approaches described herein decouple the ethylene detection event from the signal transduction event. Advantageously, a sterically encumbered metathesis precatalyst may be inert or very slow to react with substituted alkenes, yet could still react with the smallest alkene, ethylene, thereby providing a highly reactive Ru-methylidene (Ru=CH₂), as shown for example in FIG. 3A. Such in situ generated reactive species may then perform amplified signal transduction as shown, for example, in FIG. 3B. Various embodiments of one example of signal transduction are via catalytic RCM of profluorescent substrates, as shown in FIG. 3C. In FIG. 3C, ethylene is a byproduct of metathesis reactions between terminal alkenes, and this generated ethylene may be fed back into the system to activate additional portion of the latent catalyst pool.

In some embodiments, another example of signal transduction is via catalytic ring opening metathesis polymerization (ROMP) as shown in FIG. 3D. FIG. 3D illustrates steps of a ring opening metathesis polymerization (ROMP) process, in accordance with various embodiments disclosed herein. Advantageously, as shown in FIG. 3D in certain aspects, ROMP results in catalytic polymerization where over a hundred polymer units are produced for each ethylene molecule.

Some of the exemplary and advantageous features of the embodiments described herein include, and are not limited to: decoupling ethylene detection from the signal transduction, which reduces or eliminates false positive results arising from complex decomposition; amplifying ethylene detection events via catalysis as described herein; and even in the realm of catalytic olefin metathesis (e.g., a much larger field than ethylene detection) latent complexes have only been activated by light, heat, acid, and redox signals, with no explicit reports of ethylene activation.

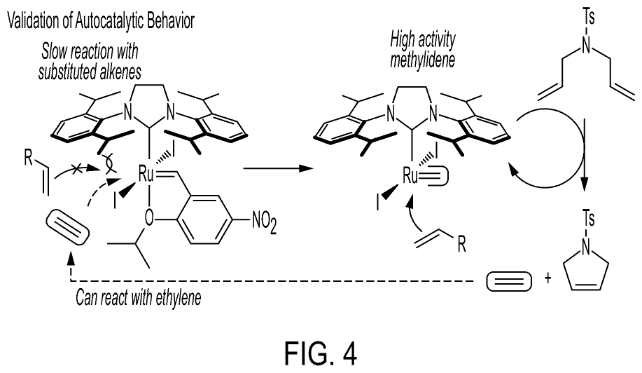

It is noted that, in some aspects, a catalyst with high latency for the substrates that can be activated by ethylene may be a factor in realizing certain embodiments of the system as described herein, such as for the ring closing metathesis (RCM) embodiments shown in FIG. 4. FIG. 4 shows an illustrative diagram of autocatalytic behavior involved in various embodiments disclosed herein. To this effect, autocatalytic behavior of metathesis catalysts may be used in the present embodiments. In various aspects, FIG. 4 shows activation of latent metathesis catalyst, providing autocatalytic and self-propagating signal transduction. As discussed herein, it is suggested that the rapid initiation in the presence of ethylene may be the result of an initial reaction between the catalysts and ethylene to generate a highly active methylidene species (see, for example, FIG. 4). In the absence of added ethylene, a sigmoidal kinetic profile may be observed (e.g., a lag phase followed by rapid acceleration). It is recognized in embodiments disclosed herein that such behavior suggests that slow initial reaction of the substrate with the complex produced ethylene as a byproduct, which could react with the sterically encumbered complex again generating a high activity methylidene catalyst.

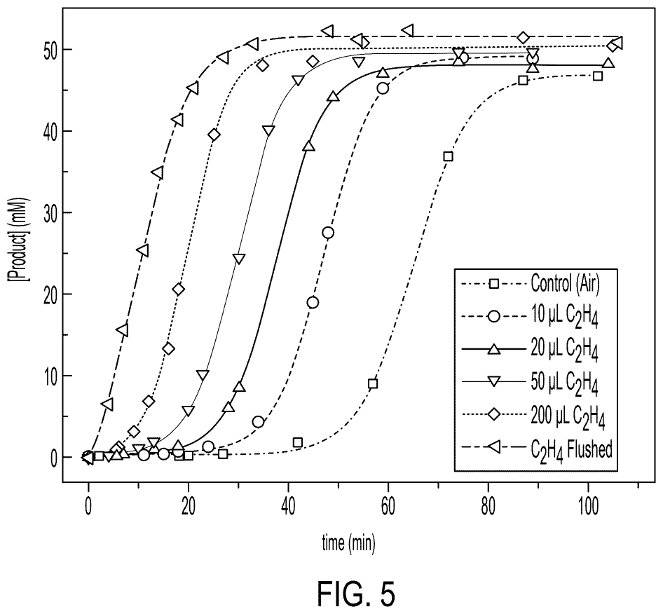

In various aspects, such behavior has been validated by monitoring by nuclear magnetic resonance spectroscopy (NMR) the RCM of TsN(allyl)₂ with varying amounts of ethylene injected into the NMR tube. FIG. 5 is an illustrative graph showing kinetic profiles for the RCM of TsN(allyl)₂ with different amounts of ethylene (C₂H₄), in accordance with various embodiments disclosed herein. In certain aspects, FIG. 5 shows kinetic profiles for the RCM of TsN(allyl)₂ with various amounts of ethylene monitored by NMR over time, demonstrating that increased amounts of ethylene (C₂H₄) modulate autocatalysis. The sigmoidal reaction profiles demonstrate an ethylene modulated dose dependence, as shown for example in FIG. 5.

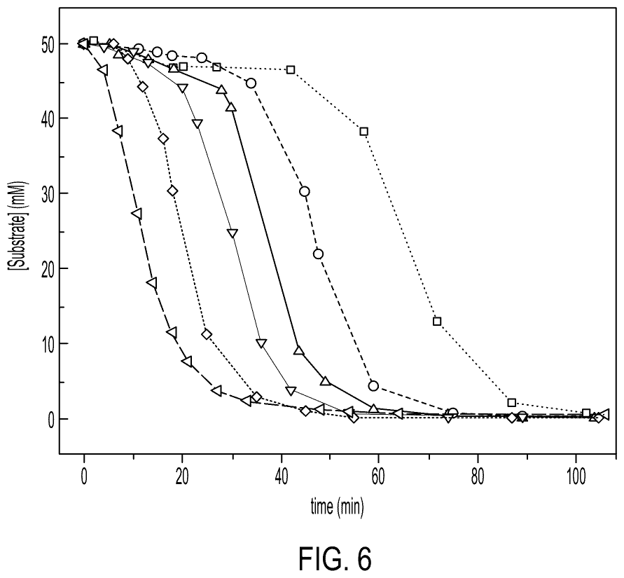

FIG. 6 is an illustrative graph showing kinetic profiles for the substrate, showing substrate consumption as a function of time, in accordance with various embodiments disclosed herein. FIG. 6 uses the same varying amounts of ethylene injected as shown by the symbols in the legend in FIG. 5, and FIG. 6 shows kinetic profiles for the substrate monitored by NMR over time, showing substrate consumption as a function of time.

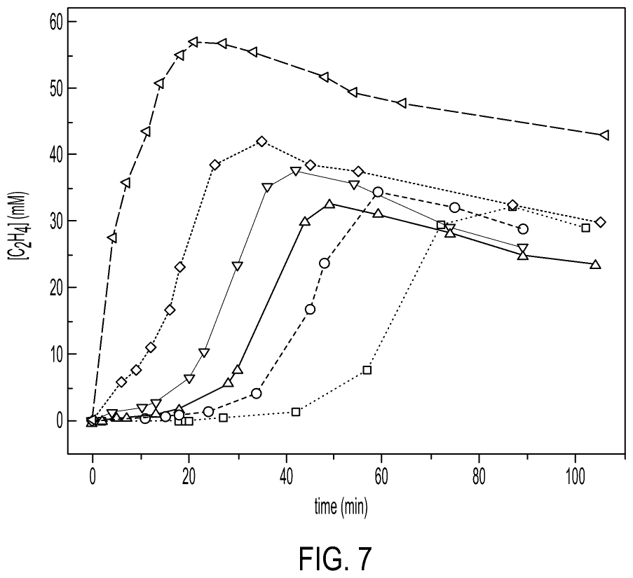

FIG. 7 is an illustrative graph showing kinetic profiles for dissolved ethylene over time, in accordance with various embodiments disclosed herein. FIG. 7 uses the same varying amounts of ethylene injected as shown by the symbols in the legend in FIG. 5, and FIG. 7 shows kinetic profiles for the dissolved ethylene monitored by NMR over time, demonstrating increased ethylene as a function of time. Monitoring dissolved ethylene shows increasing ethylene (C₂H₄) over time and clearly correlates with the acceleration of reaction velocity as measured by substrate consumption, as shown for example in FIGS. 6 and 7.

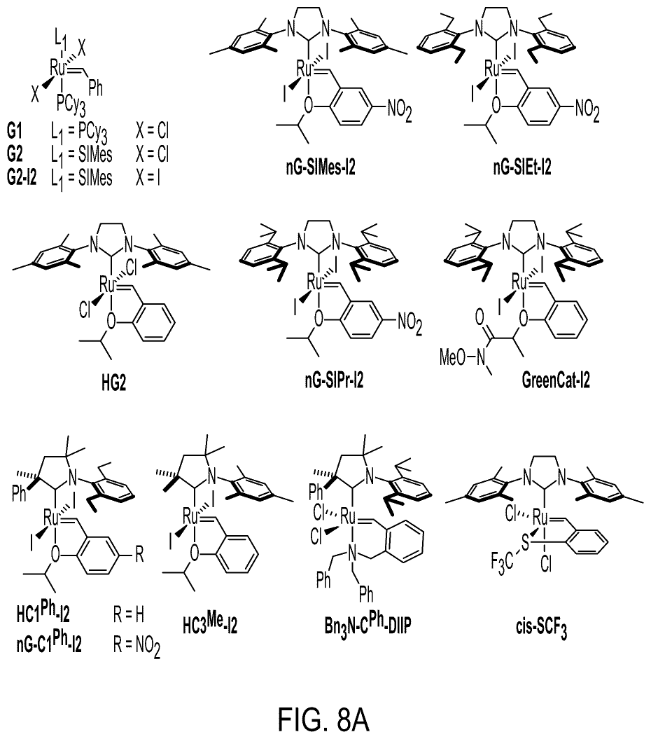

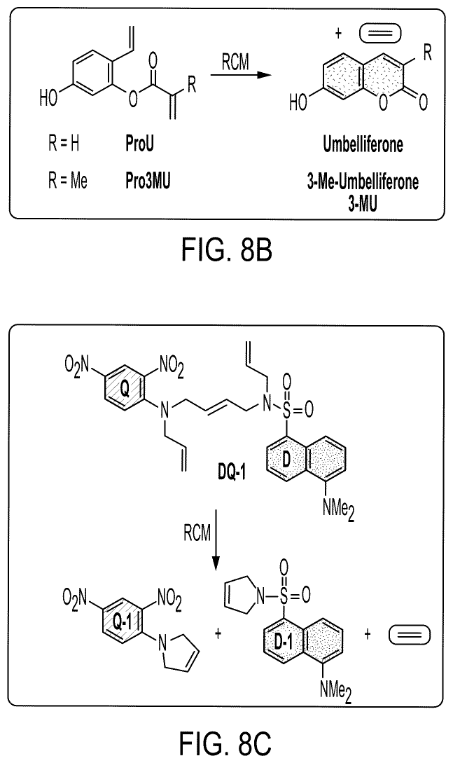

In certain aspects, taken together, the data disclosed herein support an autocatalytic behavior of the system and suggests that 12 complexes are a promising class of complexes to evaluate for ethylene activatable latent pre-catalysts. In further embodiments, a series of standard metathesis catalyst, iodide containing variants, and a select group of other catalysts were further evaluated, as shown in FIG. 8A. FIG. 8A shows an exemplary set of catalyst candidates evaluated in screening tests, in accordance with various embodiments disclosed herein. Evaluations of the latent catalysts were conducted using control reaction mixtures compared to reaction mixtures charged with 20 UL of injected ethylene. Emission scans were acquired every 15 minutes. Raw data generated from full trace scans were integrated and normalized to scans taken before ethylene addition (i.e., t=0). Table 1 shows catalyst evaluation with Pro3MU. Table 2 shows catalyst evaluation with DQ-1.

TABLE 1

| | Control | | | 20 μL C2H4 | | |
|---|---|---|---|---|---|---|
| \|Catalyst | 60 min | 90 min | 120 min | 60 min | 90 min | 120 min |
| G1 | [symbol] | [symbol] | 2.46451 | 1.59979 | 1.68357 | 1.72211 |
| G2 | 3.30951 | [symbol] | 3.54602 | 3.01917 | 3.12492 | [symbol] |
| HG2 | 1.73099 | 2.07599 | [symbol] | [symbol] | 3.16144 | [symbol] |
| G2-I2 | 1.56345 | 1.94475 | 2.29607 | 1.1148 | 1.12536 | 1.141 |
| nG-SIMes-I2 | [symbol] | [symbol] | [symbol] | 3.64352 | [symbol] | [symbol] |
| nG-SIEt-I2 | 0.98582 | 0.99418 | 0.99762 | [symbol] | 4.20244 | [symbol] |
| nG-SIPt-I2 | 0.97647 | [symbol] | 0.97063 | 3.15299 | 4.06163 | 4.65737 |
| Greencat-I2 | 0.98845 | [symbol] | 0.99243 | 1.10643 | 1.13995 | 1.19015 |
| HC1Ph-I2 | 1.02687 | 1.02775 | 1.04227 | [symbol] | [symbol] | 1.0137 |
| HC3Me-I2 | 0.99275 | [symbol] | 1.0056 | 1.55112 | 1.87414 | 2.19583 |
| nG-HC1Ph-I2 | 1.02494 | 1.0455 | 1.0644 | [symbol] | [symbol] | 1.0855 |
| Bn3N-CPh | 1.04185 | 1.06033 | [symbol] | 1.11871 | 1.23047 | 1.37691 |
| cis-SCF3 | 1.01465 | 1.0322 | 1.05443 | 1.02097 | [symbol] | [symbol] |

[symbol]

indicates data missing or illegible when filed

In Table 1, appreciable latency was observed with nG-SIMes-I2, nG-SIEt-I2, and nG-SIPr-I2. The conditions for the data collection were Pro3MU equal to 200 μM, catalyst equal to 50 μM, in toluene containing 0.4% dimethyl sulfoxide (DMSO), at 50° C. The scan parameters were λex=320 nanometer (nm), λem=335-575 nm, and excitation slit width equal to 5 nm, emission slit width equal to 10 nm.

TABLE 2

| | Control | | | 20 μL C2H4 | | |
|---|---|---|---|---|---|---|
| Catalyst | 30 min | 45 min | 60 min | 30 min | 45 min | 60 min |
| G1 | 1.16746 | 1.27174 | 1.25056 | 2.35632 | 2.74296 | [symbol] |
| G2 | 7.95001 | 11.01942 | 13.69429 | 6.91966 | 10.56099 | 13.61459 |
| HG2 | [symbol] | 3.21372 | 4.48507 | [symbol] | 4.54497 | [symbol] |
| G2-I2 | [symbol] | 1.93063 | 2.21636 | 2.12862 | 2.24363 | 2.83275 |
| nG-SIMes-I2 | [symbol] | 4.27358 | 6.53332 | 46.21647 | 53.21462 | 55.99582 |
| nG-SIEt-I2 | 1.87716 | 2.70366 | 3.86943 | 43.31601 | 56.00946 | [symbol] |
| nG-SIPt-I2 | 1.0663 | [symbol] | 1.24905 | 50.0262 | 61.95692 | [symbol] |
| GreenCat-I2 | 0.97524 | 0.97885 | [symbol] | 1.60227 | 2.12166 | [symbol] |
| HC1Ph-I2 | [symbol] | [symbol] | [symbol] | 0.94999 | 0.80516 | 1.03399 |
| HC3Me-I2 | 1.0395 | [symbol] | 1.23311 | 1.1918 | [symbol] | [symbol] |
| nG-C1Ph-I2 | 1.02115 | 1.02059 | 1.0397 | 1.60109 | 2.00946 | 2.47462 |
| Bn3N-CPh | 1.06131 | [symbol] | 0.95865 | 3.22869 | 4.4772 | [symbol] |
| cis-SCF3 | 1.02624 | [symbol] | 1.04025 | 1.03621 | 1.04418 | 1.05921 |

[symbol]

indicates data missing or illegible when filed

In Table 2, appreciable latency was observed with nG-SIMes-I2, nG-SIEt-I2, and nG-SIPr-I2. Conditions: [DQ-1]=200 μM, [Catalyst]=50 μM, in toluene, 20° C. Scan Parameters: λex=320 nm, λem=360-620 nm, excitation slit width equal to 5 nm, emission slit width equal to 5 nm.

A chemical structure for 1,3-dimesityl-4,5-dihydro-1H-imidazol-3-ium-2-ide (SIMes) is shown in Chemical Structure 5:

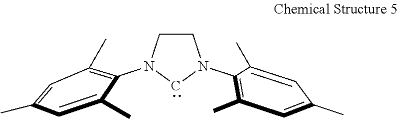

A chemical structure for 1,3-bis(2,6-diisopropylphenyl)-4,5-dihydro-1H-imidazol-3-ium-2-ide (SIPr) is shown in Chemical Structure 6:

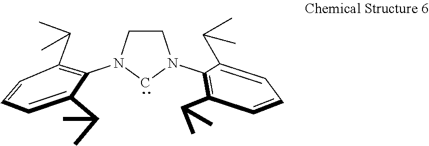

A chemical structure for 1,3-bis(2,6-diethylphenyl)-4,5-dihydro-1H-imidazol-3-ium-2-ide (SIEt) is shown in Chemical Structure 7:

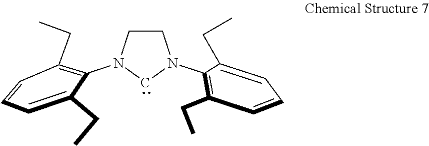

In various embodiments, nG complexes refer to ruthenium benzylidene complexes with a chelating ligand containing an isopropyl ether group and a nitro group, e.g., (2-isopropoxy-5-nitrobenzylidene) ruthenium, as shown in Chemical Structure 8:

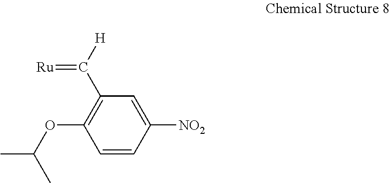

The catalysts, solvents, and substrates for use in embodiment described herein are not limited by the examples disclosed herein, and a wide range of catalysts, solvents, and substrates, and their concentrations, are compatible with the embodiments disclosed herein. For example, a wide range of catalyst and substrate concentrations are compatible with the amplificative systems disclosed herein and may be determined and/or optimized based on an expected amount of ethylene to be detected. Examples of solvents for use with embodiments of the present disclosure include and are not limited to benzene, acetone, dicholormethane, mesitylene, THF, and hexanes.

Approaches described in various embodiments herein look at substrates that cyclize to provide products with increased fluorescence, either by fluorophore synthesis or cleavage of an appended quencher. In exemplary embodiments, the catalysts were evaluated for catalyst latency and ethylene activation by monitoring the RCM of profluorescent, pro-umbelliferone (ProU) substrate, which generates fluorescent umbelliferone upon RCM (see, for example, FIG. 8B, R=H).

A chemical structure for 7-hydroxy-3-methyl-2H-chromen-2-one (3-Me-Umbelliferone; 3-MU) is shown in Chemical Structure 9:

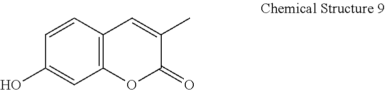

FIG. 8B illustrates an RCM process that ProU and Pro3MU undergo to provide Umbelliferone and 3-Me-umbelliferone as fluorescent products along with ethylene, in accordance with various embodiments disclosed herein. As ProU was found that it may be prone to polymerization and/or decomposition, the methacrylate analogue of ProU (e.g., Pro3MU as shown in FIG. 8B, R=Me) was used to demonstrate increased stability, particularly when stabilized with BHT (about 1000 parts per million (ppm)). Additionally, a dansyl-quencher substrate (e.g., DQ-1 in FIG. 8C) was also evaluated. FIG. 8C illustrates a relay RCM process with DQ-1 and products, in accordance with various embodiments disclosed herein.

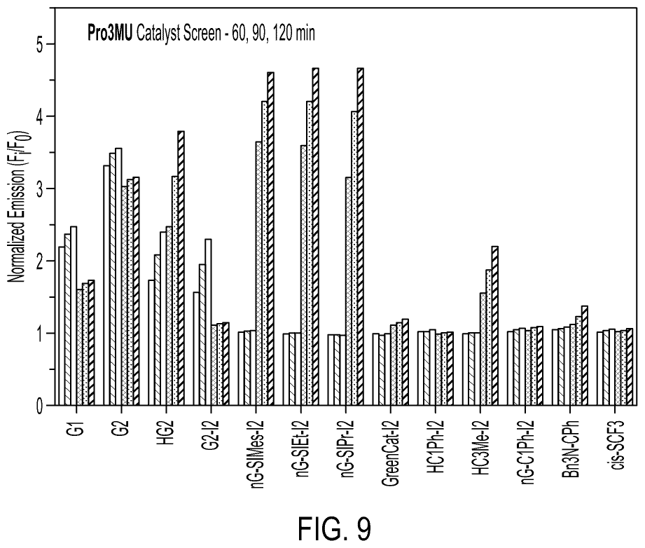

FIG. 9 shows an illustrative bar graph summarizing results of a catalyst screening process with Pro3MU, in accordance with various embodiments disclosed herein. In the examples shown in FIG. 9, the catalyst screening resulting in fluorescence response was measured with the following concentrations and temperature: [Pro3MU]=200 μM, [Catalyst]=50 μM, PhMe, 50° C. In FIG. 9, the control is shown in as the three bars on the left ( [symbol] ) for each catalyst, and the 20 μL C₂H₄ is shown as the three bars on the right ( [symbol] ) at each of 60, 90, and 120 minutes. In FIG. 9, the less reactive Pro3MU substrate was evaluated in toluene at 50° C. by monitoring fluorescence increase over time in the absence of added ethylene or exposed to 20 μL ethylene gas. Under these conditions, iodide complexes bearing saturated imidazole N-heterocyclic carbene (NHC) ligands appeared that they may be the most promising (see, e.g., FIG. 9). Little to no fluorescence response was observed in the absence of ethylene with a strong, or relatively stronger, response when exposed to ethylene. CAAC-I2 complexes were relatively slow to react or inert under these conditions, the other 12 complexes had results were unimpressive, and phosphine containing complexes (e.g., G1 and G2 in FIG. 9) performed worse in the presence of ethylene.

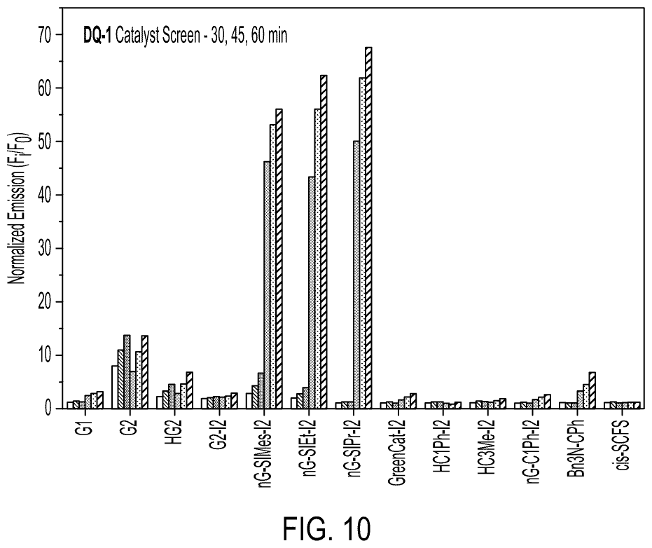

FIG. 10 shows an illustrative bar graph summarizing results of a catalyst screening process with DQ-1, in accordance with various embodiments disclosed herein. Similar patterns of catalyst reactivity were observed for DQ-1 at room temperature, although with notably faster product formation at lower substrate and catalyst concentrations (as shown, for example, in FIG. 10). This behavior may be explained by a relative lack of steric hinderance proximal to the alkene. In some embodiments, the steric and electronic nature of the substrate alkenes affects the latency and temporal response of the system. Additional modifications to the substrates can result in systems with faster or slower response as desired for a particular application.

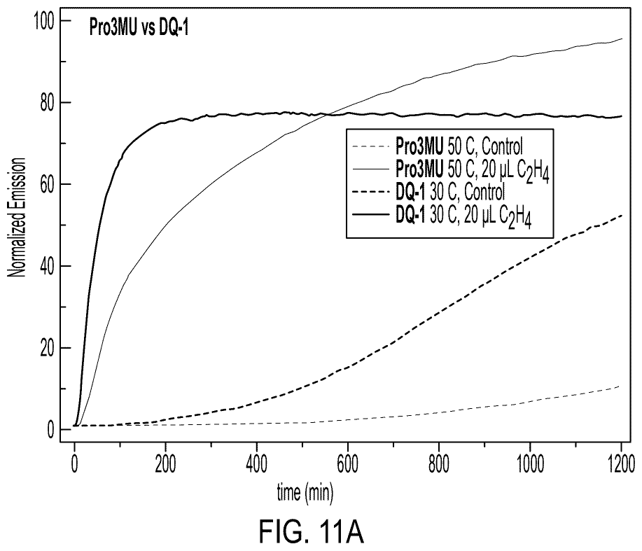

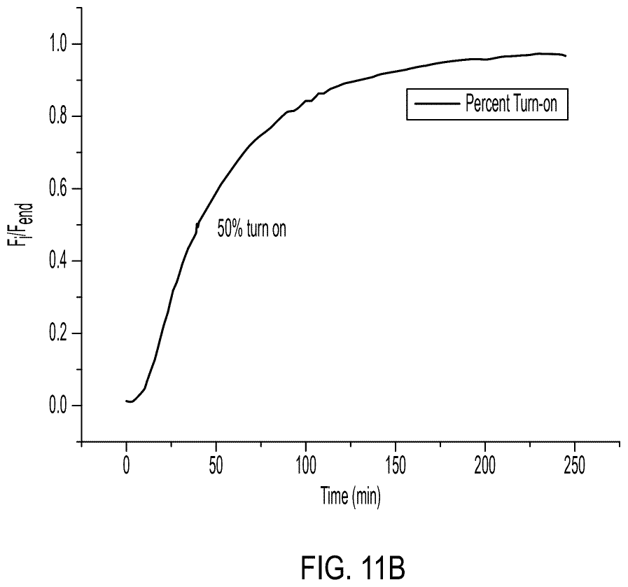

FIG. 11A is an illustrative graph comparing relative substrate response over time, in accordance with various embodiments disclosed herein. In some aspects, the disparity in signal production was highlighted by monitoring fluorescence increase over time for the two substrates at the same concentration (see, for example, FIG. 11A). FIG. 11B is an illustrative graph comparing florescence turn-on values for DQ-1 over time, in accordance with various embodiments disclosed herein. As shown in FIG. 11B, DQ-1, (exposed to 20 uL ethylene at 30° C.) reaches ^{˜}50% of turn-on within just 40 minutes, while Pro3MU (at 50° C.) requires 3 hours to reach ^{˜}50% turn-on. For example, as shown in FIG. 11B, the fluorescence intensity (Fi) over endpoint fluorescence was shown at various times for DQ-1, and DQ-1 reaches a maximum turn-on of ^{˜}78 fold. When exposed to 20 uL ethylene at 30° C., the fluorescence response reaches 39-fold after 40 minutes.

As shown, for example, in the illustrative embodiments of FIG. 11, the kinetic profile of Pro3MU appears to lack the classic sigmoidal shape associated with autocatalysis, possibly due to slow turn-over of an arylogous Fisher carbene intermediate. The tradeoff for faster kinetics of DQ-1 appears that it may be a lowered degree of latency. Advantageously, even in the absence of added ethylene, an increase in fluorescence was eventually observed, which highlights the autocatalytic nature of this system. For example, slow background reactivity generates ethylene, which was then capable of converting more of the latent catalyst pool into the reactive methylidene.

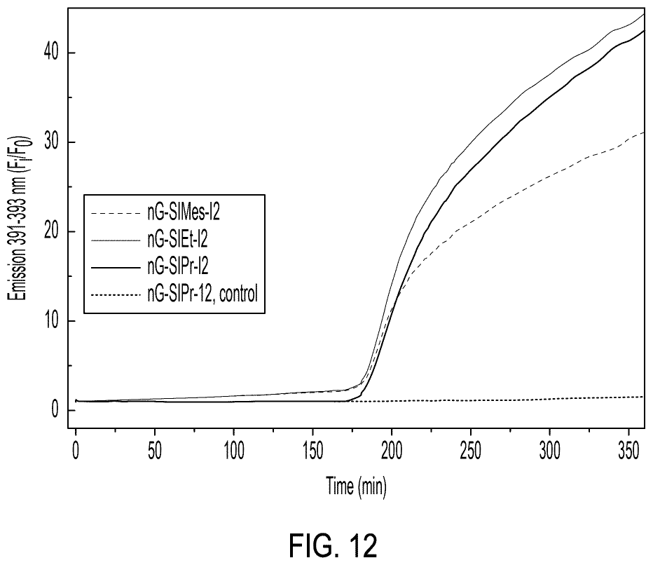

FIG. 12 is an illustrative graph summarizing the evaluation results of extended Pro3MU latency with 12 catalysts, in accordance with various embodiments disclosed herein. In the data shown in FIG. 12, iodinated nitroGrela-NHCs demonstrated a similar response, and systems were tested for extended latency using 50 μL injected ethylene dispensed at 180 min with conditions of Pro3MU equal to 200 μM, catalyst equal to 50 μM, in toluene containing 0.4% DMSO, and 50° C. The scan parameters were λex=320 nm, λem=335-575 nm, excitation slit width equal to 5 nm, and emission slit width equal to 10 nm. FIG. 12 tracks different latent catalyst systems showing normalized emission of Fᵢ/F₀ for the conversion of Pro3MU to 3MU (illustrative embodiments of the conversion are shown in Chemical Reaction 1).

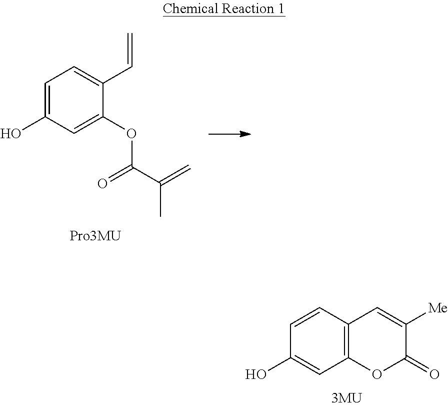

As shown in the various embodiments of this example, a closer examination of Pro3MU with the promising iodo complexes nG-SIMes-I2, nG-SIEt-I2, and nG-SIPr-I2 over time indicated extended latency of all three complexes at 50° C. (see, for example, FIG. 12). However, nG-SIPr-12 may have essentially no fluorescence increase over this time, and for another 180 minutes, in contrast to a modest fluorescence response for the SIMes and SIEt 12 complexes. This discrepancy may be further exacerbated at higher temperatures. Injection of 50 microliter (uL or uL) of ethylene at t=180 minutes resulted in a larger fluorescence increase for all three complexes. In various aspects, the lesser turn-on of nG-SIMes-I2 under these conditions could indicate decomposition of the precataylsts, ethylene adducts, or a greater propensity for unproductive ethylene self-metathesis for this less sterically protected complex.

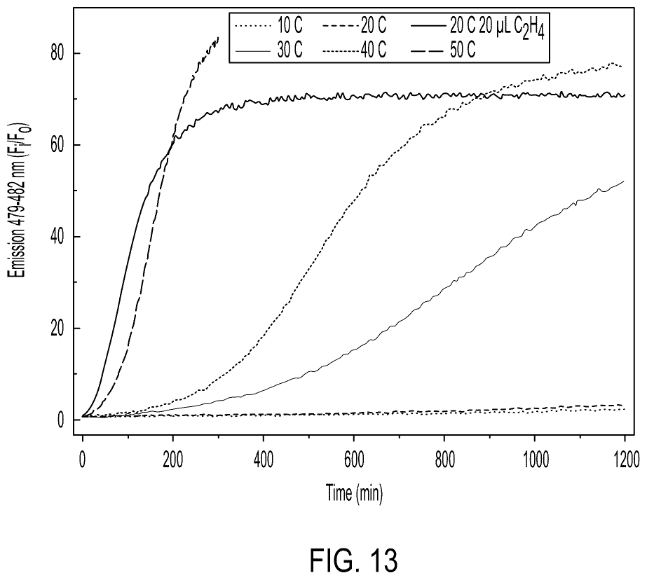

FIG. 13 is an illustrative graph summarizing the influence of temperature on DQ-1 latency, in accordance with various embodiments disclosed herein. In these illustrative embodiments, sufficient latency of DQ-1 can be obtained with nG-SIPr-I2 by controlling the cuvette temperature at 20° C., while maintaining robust response to 20 μL C₂H₄, as shown for example in FIG. 12. Higher temperatures may result in significant catalysts initiation in the absence of ethylene. Overall, in these illustrative examples, the limited latency of DQ-1 and the autocatalytic nature of the system resulted in less consistent control runs. These results demonstrate that substrate structure can be used to tune the signal generation properties of the system. In the exemplary system demonstrated herein, the Pro3MU substrate was selected, owing to its robust latency with nG-SIPr-I2.

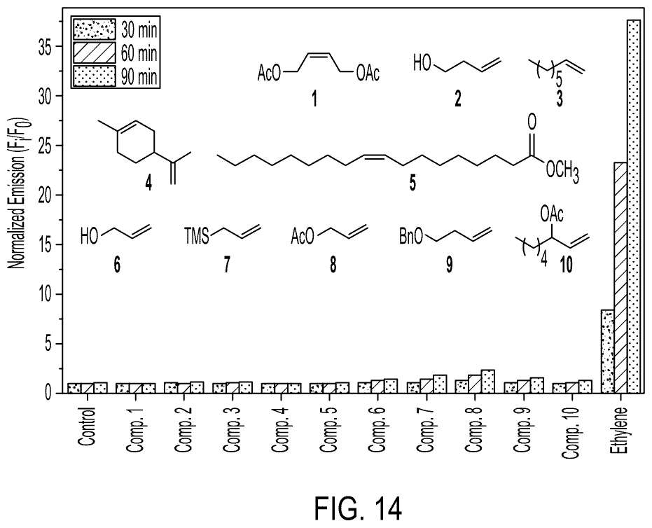

FIG. 14 is an illustrative bar graph summarizing the results of the evaluation of selectivity for C₂H₄ compared to other substrates, in accordance with various embodiments disclosed herein. In certain embodiments, the Pro3MU/nG-SIPr-I2 system shows high selectivity for ethylene (see, for example, FIG. 14). In some aspects, internal alkenes, trisubstituted and Z-disubstituted showed no ability to initiate the catalyst system. A very limited response was observed with some small terminal alkenes; however, the response was over a magnitude less as compared to ethylene. Amplified response was not observed from other substrates, so the amplitude of ethylene selectivity actually increases over time.

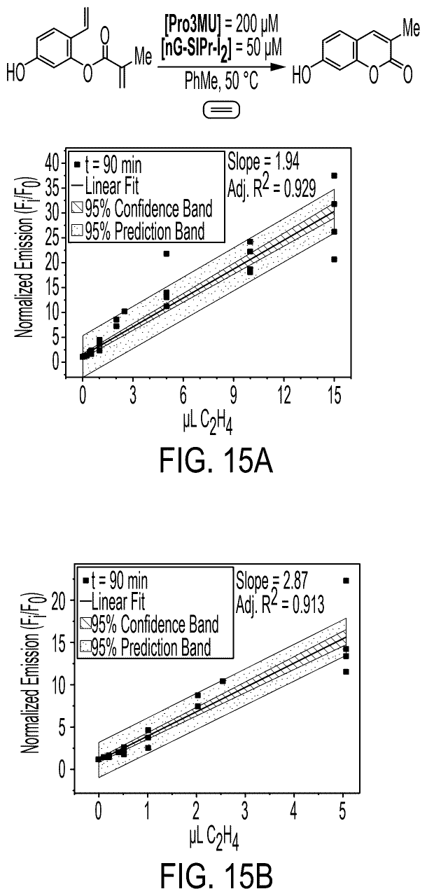

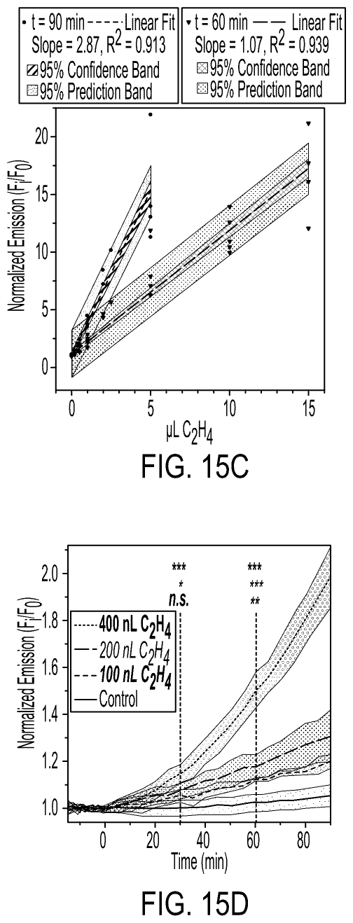

FIGS. 15A-D show, for example, limits of detection experiments with injected ethylene in accordance with various embodiments disclosed herein. For example, FIG. 15A shows an illustrative line region for limit of detection experiments with injected ethylene (e.g., linear region from 0-15 uL at t=90 minutes); FIG. 15B shows an illustrative line region for limit of detection experiments with injected ethylene (e.g., linear region from 0-5 uL at t=90 minutes); FIG. 15C shows illustrative combined regions of linearity for limit of detection experiments with injected ethylene (e.g., combined regions of linearity at t=60 minutes and t=90 minutes); and FIG. 15D shows illustrative fluorescence intensity over time at low volumes of ethylene for limit of detection experiments with injected ethylene, in accordance with various embodiments disclosed herein.

As described in some embodiments disclosed herein, the combination of latency with ethylene triggered auto-catalysis advantageously can result in a remarkably sensitive system. In various embodiments, as an initial assessment of sensitivity, septum sealed cuvettes containing the reaction mixture were injected with ethylene-nitrogen gas mixtures. Due to the small volumes of gas being injected, it may be useful to consider the amount (volume or moles) of ethylene detected rather than concentration. A linear response was observed early in the reaction time. In these linear regimes, the Limit-of-Detections (LODs) may be calculated to be 109 nL at 60 minutes (0-15 μL) and 50 nL at 90 minutes (0-5 μL), as shown for example in FIGS. 15A-C. Thus, in certain aspects, monitoring fluorescence over time may clearly demonstrate sensitivity with a significant response observed down to 100 nL ethylene (see, for example, FIG. 15D). In various aspects, the high sensitivity of ADE is well below the sensitivity needed to detect ethylene levels from plants (0.1-10 μL/L) and into the range reported for ethylene in exhaled breath from mammals (1-1000 nL/L).

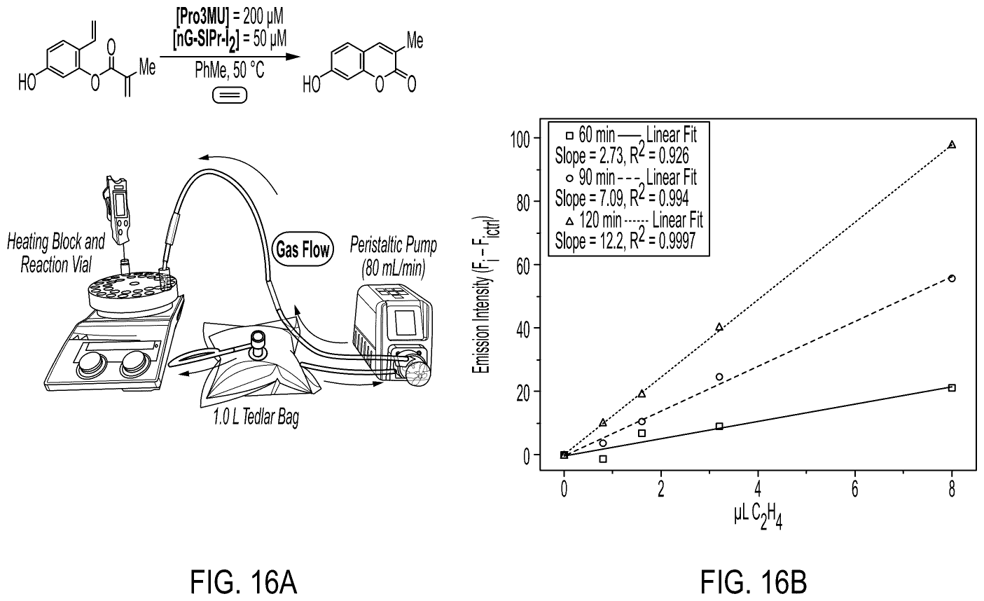

FIG. 16A is an illustrative example flow system for larger volume dilute ethylene sampling in accordance with various embodiments disclosed herein. FIG. 16B shows an illustrative demonstration of fluorescence response from a flow system in accordance with various embodiments disclosed herein. Current commercial systems for ethylene detection require high flow and/or large volumes. For example, laser-based systems capable of nL/L sensitivity with gas scrubbing operate at ˜1-5 L/h. Another system, a handheld electrochemical ethylene sensor that is used for in-house comparison, samples at 300 mL/min (0-100 μL/L sensitivity. To better mimic these techniques, a flow assay was used to sample dilute ethylene gas mixtures; an illustrative example of such a system is shown in FIG. 16A. In this illustrative system, a peristaltic pump was used to percolate samples through the ADE reaction mixture for 2 minutes at 80 mL per minute, and fluorescence measurements were acquired at set timepoints. Although the flow system incompletely captures ethylene for ADE, an obvious response was observed into the nL range as shown, for example, in FIG. 16B and discussed herein. In certain aspects, FIG. 16B shows a demonstration of fluorescence response from this system at 60 minutes, 90 minutes, and 120 minutes. To collect the data shown in FIG. 16B, dilutions of ethylene were mixed using a GB103 gas mixer (MCQ Instruments) and dispensed into 1.0 L tedlar bags, and reaction mixtures were prepared and added to septa-sealed 2-dram vials equipped with a stir bar. Gas mixtures were bubbled into samples for 2 minutes with a 23 G needle and equivalent gauge needle plunged through the septa to vent. Samples were heated to 50° C. for 60, 90, and 120 minutes, and endpoint emission scans were acquired upon completion of the run. Fluorescent intensities were averaged across the observed emission wavelengths of each sample and the average fluorescence intensity of control runs were subtracted.

To collect the example data shown in FIG. 16B for the flow sampling response with a Pro3MU substrate, the conditions were Pro3MU equal to 200 μM, nG-SIPr-I2 equal to 50 μM, in toluene containing 0.4% DMSO, at 50° C. with scan parameters of λex=320 nm, λem=388-393 nm, excitation slit width equal to 10 nm, emission slit width equal to 10 nm, and each data point represents an individual technical replicate. Results showing exposure of Pro3MU substrate to large volumes of low concentration ethylene are shown in Table 3.

TABLE 3

| | uL C2H4 | | | |
|---|---|---|---|---|
| [Ethylene] in | (2 min at | Fi—Fi(cntrl) at Reaction Time | | |
| sample bag | 80 mL/min) | 60 min | 90 min | 120 min |
| Breathing Air | 0 | 0 | 0 | 0 |
| 5 ppm | 0.8 | −1.312 | 3.583 | 9.963 |
| 10 ppm | 1.6 | 6.703 | 10.329 | 19.018 |
| 20 ppm | 3.2 | 8.985 | 24.541 | 40.147 |
| 50 ppm | 8 | 21.057 | 55.607 | 97.651 |

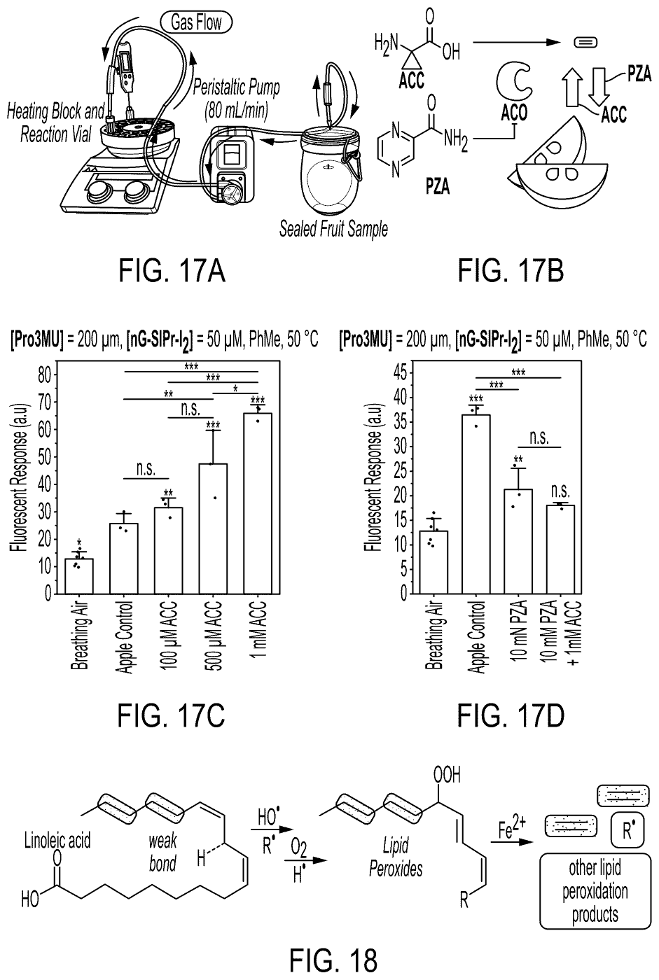

FIG. 17A shows an experiment demonstrating endogenous ethylene detection from apples; FIG. 17B is an illustrative diagram showing the influence of 1-aminocyclopropane-1-carboxylic acid (ACC) and pyrazinamide (PZA) on ethylene production; FIG. 17C is an illustrative bar graph of average fluorescence response to increasing amounts of ACC; and FIG. 17D is an illustrative bar graph of average fluorescence response to inhibition of ACC oxidation (ACO) with PZA, in accordance with various embodiments disclosed herein.

In some aspects, to demonstrate the utility of the high sensitivity of these systems, ethylene from apple slices were detected using the catalytic amplification approach described herein. In an exemplary experiment, apple slices were placed in sealed glass jars, treated as indicated, a left for a set period of time 24 hours. After this time the air in the jars was sampled for 2 minutes at a set rate of 81 mL per minute, with the air bubbled through vial containing the optimized Pro3MU system, as shown for example in FIG. 17A. Amplificative ethylene detection was allowed to proceed for set periods of time at which point fluorescence was measured. Here the vehicle control (water) represents basal ethylene levels. Ethylene levels can be increased in a dose dependent manner by addition of 1-aminocyclopropane-1-carboxylic acid (ACC), the native precursor to ethylene in plants. Addition of pyrazinamide (PZA), a known inhibitor of ACO, almost completely inhibits ethylene production and reduces ethylene production when samples were also treated with ACC. Exemplary results of these additions are shown in FIG. 17B.

In an illustrative example, apple slices were treated with purified water only, to represent the basal level of ethylene produced by the apple samples (one such example of a sampling jar is shown in FIG. 17A). After treating the apples with the purified water, 10 mM PZA was added, upon which a knockdown in signal relative to the control was observed, thus confirming the observation of basal levels of ethylene. In contrast, the treatment of the apple slices with ACC (1-10 mM) was observed to result in a significant increase in the amount of ethylene detected, as expected. Simultaneous treatment of the apple slices with PZA has been observed to attenuate the ethylene detection signal back to basal levels. Exemplary results are shown in FIGS. 17C-D.

Further, as discussed herein, ethylene has been implicated as a biomarker of oxidative stress in mammalian systems arising from lipid peroxides via a non-enzymatic pathway. Owing to the method's high sensitivity, laser photoacoustic spectroscopy (LPAS) has been used to provide real-time monitoring of ethylene in exhaled breath. Concentrations of ethylene may be suggested to be in the range of 10-100 nL C₂H₄/L (10-100 parts per billion (ppb)) for individuals under oxidative stress conditions.

FIG. 18 shows an illustrative proposed route to ethylene from lipid peroxides, in accordance with various embodiments disclosed herein. To assess the potential of the system disclosed herein to detect ethylene from lipid peroxides, polyunsaturated fatty acid methyl esters were subjected to conditions previously reported to result in ethylene production. A simplified diagram of a proposed process is shown by way of example in FIG. 18.

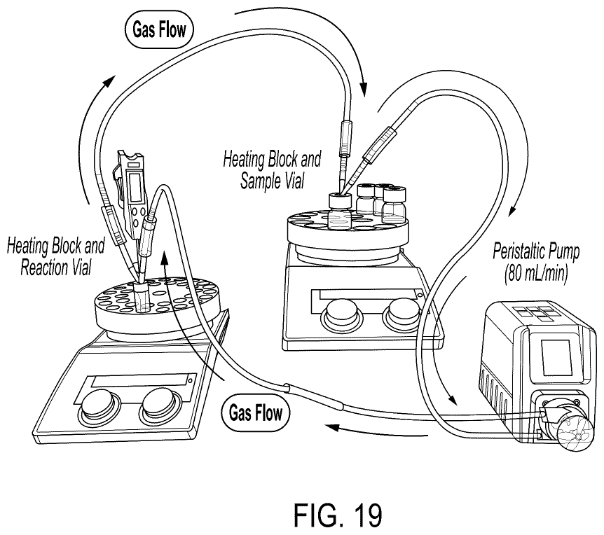

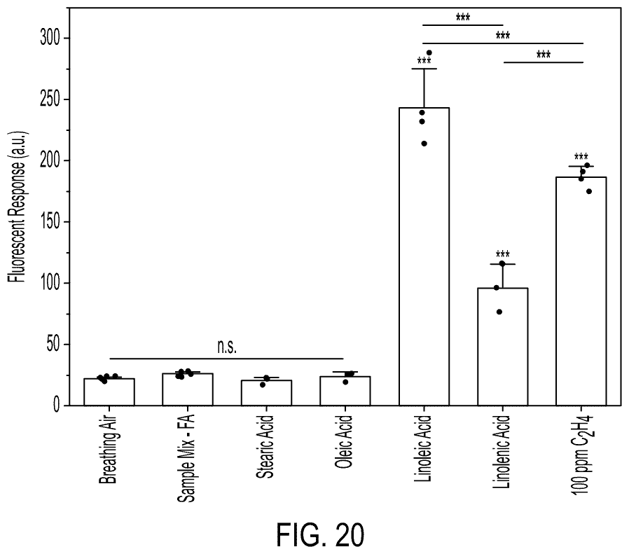

FIG. 19 is an illustrative closed loop sampling set-up employed in lipid peroxidation experiments in accordance with various embodiments disclosed herein. In an example, samples were prepared in 20 mL reaction vials containing breathing air or 100 ppm ethylene as negative and positive controls respectively. In another vial 0.5 mL of 180 mM linoleic acid, 0.5 mL of 10 mM CuSO₄, and 0.5 mL 20 mM ascorbic acid in 1% Brij-35/PBS was aliquoted (with additional 1% Brij-35/PBS added to reach target volume). Samples were stirred overnight in heating mantle at 37° C. After stirring at 37° C. overnight, ethylene in the headspace was measured using a closed-loop sampling system, as shown by way of example in FIG. 19, for two minutes at a set rate of 80 mL/min, with the air bubbled through vial containing the optimized Pro3MU system. Exemplary results are shown in FIG. 20. In various embodiments, the time and gas flow of the systems disclosed herein may be determined and/or optimized as needed for various applications.

FIG. 20 is an illustrative bar graph showing the detection of ethylene from peroxidation and fragmentation of linoleic acid and linolenic acid, in accordance with various embodiments disclosed herein. In certain aspects, FIG. 20 summarizes the advantageous results of evaluation of the applicability of the high sensitivity, amplificative ethylene detection approach to the detection of ethylene in exhaled breath.

In various embodiments, described herein is a synthetic amplificative system that has signal transduction amplified by an organometallic catalytic process (such as RCM) and a self-propagating process (ethylene feedback). Various improvements to the herein-described system are contemplated, such as through catalyst and substrate optimization to enable ethylene directly in models of oxidative stress, and these contemplated improvements are considered to be a part of the present disclosure.

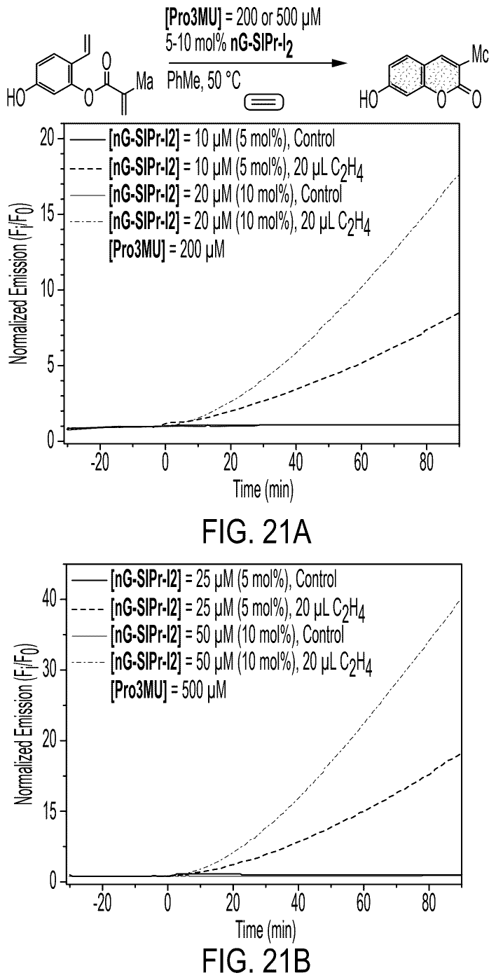

FIGS. 21A and 21B show normalized emission over time for Pro3MU for concentrations of catalyst and substrate, in accordance with various embodiments disclosed herein. Time course experiments were conducted at the concentrations shown in FIGS. 21A and 21B. To collect the data, cuvettes were charged with 0 or 20 μL ethylene post thermal stabilization, and emission scans were acquired every 5 minutes to a total run time of 90 minutes. Fluorescent intensities were averaged across the observed emission wavelengths of each sample and normalized to their respective t=0. Due to a dip in observed intensity noted during thermal stabilization, t=0 values were generated by averaging the emission values recorded five minutes prior to ethylene injection. The conditions were Pro3MU equal to 200 μM, nG-SIPr-I2 equal to 10 UM or 20 μM (5 or 10 mol %), in toluene containing 0.4% DMSO for FIG. 11A. The conditions were Pro3MU equal to 500 μM, nG-SIPr-I2 equal to 25 UM or 50 μM (5 or 10 mol %), in toluene containing 1% DMSO for FIG. 11B. The conditions for FIGS. 21A and 21B included λex=320 nm, λem=388-393 nm, excitation slit width equal to 5 nm, emission slit width equal to 10 nm.

In these exemplary embodiments, the catalyst concentration was varied from 10-50 micromolar (uM or μM) and the substrate concentration was varied from 200-500 μM. In various aspects, higher and lower concentrations of both substrate and catalyst are possible for embodiments disclosed herein. Generally, in some examples and as shown in FIGS. 21A and 21B, the higher the concentrations the faster the signal response was observed.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which the embodiments of this disclosure are entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”—whether explicitly discussed or not—and, conversely, were there only disclosure of the act of “protruding”, such a disclosure should be understood to encompass disclosure of a “protrusion”. Such changes and alternative terms are to be understood to be explicitly included in the description.

What is claimed is:

1. A method for ethylene detection, comprising:
activation of an olefin metathesis catalyst by initial ethylene molecules to produce signal molecules and secondary ethylene molecules, wherein the ethylene comprises the initial ethylene molecules and the secondary ethylene molecules; and
detecting the ethylene.

2. The method of claim 1, wherein the detecting the ethylene is based on the secondary ethylene molecules.

3. The method of claim 2, wherein the detecting the ethylene is further based on self-propagation.

4. The method of claim 1, further comprising:
in-situ generation of latent units of the olefin metathesis catalyst that performs multiple cycles of substrate conversion to obtain an amplified signal of the ethylene.

5. The method of claim 4, wherein an amount of the amplified signal corresponds to an amount of the secondary ethylene molecules.

6. The method of claim 1, wherein more of the signal molecules are produced than the secondary ethylene molecules.

7. The method of claim 1, further comprising: converting, using ring closing metathesis, non-fluorescent molecules to fluorescent molecules along with at least some of the secondary ethylene molecules.

8. The method of claim 7, wherein the secondary ethylene molecules generate additional ethylene, and wherein the additional ethylene activates latent units of the olefin metathesis catalyst to provide an autocatalysis process.

9. The method of claim 4, wherein the substrate conversion occurs by at least one of ring closing metathesis reactions, ring opening metathesis polymerization reactions, cross metathesis reactions, and acyclic diene metathesis polymerization reactions.

10. The method of claim 4, further comprising signal transduction during the substrate conversion.

11. The method of claim 10, wherein the signal transduction occurs via at least one of profluorescent ring closing metathesis reactions and induced polymerization reactions.

12. The method of claim 1, further comprising: catalytic signal transduction using at least one mechanism comprising: fluorescence reactions, colorimetric reactions, polymerization, and electrochemical reactions.

13. The method of claim 1, further comprising: after the activation, initiating ring opening metathesis polymerization using latent units of the olefin metathesis catalyst.

14. The method of claim 1, further comprising: polymerization, wherein the polymerization accelerates the production of the secondary ethylene molecules.

15. The method of claim 14, wherein the polymerization is translated into at least one other signal transduction mechanisms comprising fluorescence reactions, luminescence reactions, and colorimetric reactions.

16. A system for ethylene detection, comprising:
providing an olefin metathesis catalyst;
activating the olefin metathesis catalyst using initial ethylene molecules to produce signal molecules and secondary ethylene molecules, wherein the ethylene comprises the initial ethylene molecules and the secondary ethylene molecules; and
detecting the ethylene.

17. A method for ethylene amplification, comprising:
activation of an olefin metathesis catalyst by initial ethylene molecules to produce signal molecules and secondary ethylene molecules.

18. The method of claim 17, further comprising: detecting ethylene after the activation, wherein the detecting the ethylene is based on the secondary ethylene molecules.

19. The method of claim 18, wherein the detecting the ethylene is further based on self-propagation.

20. The method of claim 17, further comprising:
in-situ generation of latent units of the olefin metathesis catalyst that performs multiple cycles of substrate conversion to obtain an amplified signal of the ethylene.
